(19) United States
(12) Patent Application Publication
(10) Pub. No.: US 2015/0148249 A1
(43) Pub. Date: May 28, 2015

(54) METHOD OF IMPROVING THE SENSITIVITY OF COMPETITIVE IMMUNOASSAY

(71) Applicant: SHENZHEN BIOEASY BIOTECHNOLOGY CO., LTD, Shenzhen (CN)

(72) Inventors: Hai Zhu, Shenzhen (CN); Xiqing Li, Shenzhen (CN); Jinfeng Li, Shenzhen (CN)

(73) Assignee: SHENZHEN BIOEASY BIOTECHNOLOGY CO.,LTD., Shenzhen (CN)

(21) Appl. No.: 14/412,681

(22) PCT Filed: Jul. 4, 2013
(86) PCT No.: PCT/CN2013/078772

§ 371 Date: Jan. 4, 2015

(30) Foreign Application Priority Data
Jul. 6, 2012 (CN) 201210233377.2

(51) Int. Cl. G01N 33/543

(52) U.S. Cl. CPC G01N 33/54306; G01N 33/54393
USPC 506/9; 436/501; 435/7.93

(57) ABSTRACT

The present invention discloses a method improving the sensitivity of competitive immunoassay, mainly, the small molecular substances for detection first react with the antibody/receptor sufficiently, then react with the second molecules, which locates on the solid phase and participates the competitive reactions, in a short time, which may greatly improve the sensitivity of competitive immunoassay. The present invention owns an easy operation method and a low cost, does not require expensive equipments or professional technological personnel, also, it owns a high sensitivity and adapts to a plurality of immunoassay methods.

### CROSS-REFERENCES TO RELATED APPLICATIONS

This application claims the priority of Chinese patent application no. 201210233377.2, filed on Jul. 6, 2012, the entire contents of all of which are incorporated herein by reference.

### FIELD OF THE INVENTION

The present invention relates to the field of biomedical detection technology area, and more particularly, to a method of improving the sensitivity of competitive immunoassay.

### BACKGROUND

Immunological detection method is an experimental method which applies immunology theory to design a series of experiments that detect antigens, antibodies and immune cells as well as cytokines they excreted. Antigens specifically bind antibodies through the complementariness in spatial configurations between antigenic determinants on its surfaces, and hyper variable regions in antibody molecules. The same antigen molecule may have a plurality of different antigenic determinants and if two different antigen molecules own one or more the same antigenic determinants, then cross reactions could happen when they are reacting with antibodies.

Except for the complementariness in spatial configurations, combinations of antigens and antibodies are mainly due to the non-covalent methods on both molecules' surfaces, including hydrogen bonds, electrostatic forces, Van der Waals forces and hydrophobic bonds, etc. The combined immune complexes could still get dissociated under a certain conditions, and get back to their free states of both antigens and antibodies. After dissociations, both antigens and antibodies will still keep their original properties. The dissociation degree of the antigen-antibody complex mainly depends on the complimentary degrees of the three dimensional configurations between the hyper variable regions of the specific antibody molecules and the according antigenic determinants. A higher complimentary degree means a closer intermolecular distance, thus a higher interaction force, causing a more solid combination between both parts, and a harder dissociation; otherwise, dissociations could be easier to happen.

Small molecular antigens or haptens, due to less epitope domains, can't be measured by the sandwich method; they can only be detected by competitive immunoassay methods. While the present methods to detect small molecular antigens mainly include Enzyme-Linked ImmunoSorbent Assay (ELISA) test, immunochromatographic test, instrumental analysis and more, wherein, the ELISA test has been widely applied, due to its specificity, sensitivity and low detection cost; the immunochromatographic test is simple and easy to apply, but its low accuracy has also limited its applications; while the instrumental analysis method is accurate and reliable, but it requires expensive equipments and professional technical personnel, which has limited its applications, it is mainly used for confirmatory tests.

Therefore, the prior art needs to be improved and developed.

### BRIEF SUMMARY OF THE DISCLOSURE

The technical problem to be solved in the present invention is, aiming at the defects of the prior art, providing a method of improving the sensitivity of competitive immunoassay, in order to solve the problems in the prior art, that the sensitivity of the present immunoassay methods is relatively low, and not suitable for high-sensitivity detections.

The technical solution of the present invention to solve the said technical problems is as follows:
a method of improving the sensitivity of competitive immunoassay, wherein, the steps of the said method of improving the sensitivity of competitive immunoassay are as follows:
pre-reaction: a substances for detection pre-react with the receptors/antibodies;
re-reaction: a reaction products collected from the pre-reaction process react with a second molecules locating on a solid phase and attending the competitive reactions;
during the said re-reaction process, the reaction product collected from the pre-reaction process is divided into one or more portions, then reacting with the said second molecules respectively.

The said method of improving the sensitivity of competitive immunoassay, wherein, during the re-reaction process, the said pre-reaction products react with the said second molecules in a way of flowing through the said solid phase surfaces.

The said method of improving the sensitivity of competitive immunoassay, wherein, during the said re-reaction process, the reaction time between each portion of the pre-reaction product and the said second molecules is within 20 minutes.

The said method of improving the sensitivity of competitive immunoassay, wherein, during the said re-reaction process, the reaction time between each portion of the pre-reaction product and the said second molecules is within 5 minutes.

The said method of improving the sensitivity of competitive immunoassay, wherein, after the reaction between each portion of the pre-reaction product and the said second molecules is finished, the post reaction liquid is removed.

The said method of improving the sensitivity of competitive immunoassay, wherein, the reaction temperature of the said pre-reaction process is between 0° C. and 65° C., and the reaction time lasts for 0-3 hours.

The said method of improving the sensitivity of competitive immunoassay, wherein, the reaction time of the said pre-reaction process lasts for 30 minutes.

The said method of improving the sensitivity of competitive immunoassay, wherein, the said second molecules refer to the conjugates formed by a small molecules participating in the competitive reactions and a carrier proteins.

Benefits: the present invention enters on the reaction mode, provides a method improving the sensitivity of competitive immunoassay, wherein, the small molecule substances for detection first react with the antibody/receptor sufficiently, then react in a short time with the second molecules, which locates on the solid phase and participates in the competitive reactions, this method may greatly improve the sensitivity of competitive immunoassay. The present invention owns an easy operation method and a low cost, does not require expensive equipments or professional technological personnel, however, it owns a high sensitivity thus adapts to a plurality of immunoassay methods.

### DETAILED DESCRIPTION

The present invention provides a method of improving the sensitivity of competitive immunoassay. In order to make the purpose, technical solution and the advantages of the present invention clearer and more explicit, further detailed descriptions of the present invention are stated here, referencing to some embodiments of the present invention. It should be understood that the detailed embodiments of the invention described here are used to explain the present invention only, instead of limiting the present invention.

Owning a relatively low sensitivity thus not suitable for high-sensitivity detections is a common problem to immunological detections; therefore, people have developed a plurality of signal amplification methods for immunological detections, including: enzyme labeling, fluorescent labeling with rare earth chelate, radiolabeling and more, to enhance the detection signals. While the present invention enters on the reaction mode, provides a method to improve the sensitivity of competitive immunoassay, wherein, a small molecule substances for detection will first react with a antibody/receptor sufficiently, then react in a short time with another molecule, which locates on the solid phase and participates the competitive reactions, this may greatly improve the sensitivity of competitive immunoassay.

Specifically, the steps of the said method of improving the sensitivity of competitive immunoassay are as follows:
pre-reaction: a substances for detection pre-react with a receptors/antibodies;
re-reaction: a reaction products collected from the pre-reaction process react with a second molecules locating on a solid phase and attending the competitive reactions.

Wherein, the said pre-reaction means that the substances for detection will first react with the receptors/antibodies sufficiently, that is, if the said substances for detection are antigens, they will react with their according antibodies sufficiently; and if the said substances for detection are complement, they react with their according receptors sufficiently.

The said pre-reaction, specifically, lasts for 0-3 hours reaction between 0° C. and 65° C., and the reaction time is preferably chosen to be 30 minutes.

The said re-reaction, specifically, means dividing the reaction products collected from the pre-reaction process into one or more portions, before respectively reacting with the said second molecules attending to the competitive reaction. Preferably, the reaction product collected from the pre-reaction process may be divided equally into 2˜6 portions, then each portion of the reaction product is put into reactions with the said second molecules locating on the solid phase, respectively. The said reaction product collected from the pre-reaction process is preferably chosen in the way of flowing through the solid phase surfaces, which have the second molecules attending the competitions coated. Experiments have proven that, the said pre-reaction product reacting with the said second molecules, in the way of flowing through the said solid phase surfaces, could improve the sensitivity of competitive immunoassay. Wherein, the said second molecules attending the competitive reaction refer to the conjugates formed by small molecules participating in the competitive reactions and the carrier proteins. And the said small molecules participating in the competitive reactions specifically refer to the complement or antigens according to the said receptors.

During the said re-reaction process, the reaction time between each portion of the pre-reaction product and the said second molecules is controlled to be within 20 minutes, preferably, within 5 minutes. The main improvement of the present invention is that, the pre-reaction product is divided into one or multiple portions during the said re-reaction process, before reacting in a short time with the second molecules attending the competitive reactions. A plurality of experiments has proven that, the sensitivity of the competitive immune reaction can be improved significantly. While the post reaction liquid, after the reactions between each portion of the said pre-reaction product and the said second molecules, shall be removed, before taking another portion of pre-reaction product to react with the said second molecules. By this way, it is possible to reduce the effects of the reaction liquid leftover to the next reaction, thus improve the sensitivity of the immunoassay detections.

Before carrying out the said re-reaction step, the said method of improving the sensitivity of competitive immunoassay further includes the following steps:
Transfer the said second molecules onto a solid phase.

For example, apply the said small molecules attending the competitive reactions to coat the ELISA plates. Further, after the said small molecules attending the competitive reactions have coated the ELISA plates, blocking solutions may be applied to enclose the said solid phase, so bindings between the antibodies and nonspecific antigens can be effectively avoided, which, otherwise, could cause a false positive result, therefore this has made the detection results more accurate. While the said blocking solution may be BSA, skim milk powder and more. The said enclosing step may be described as adding blocking solutions and incubating at 37° C. for 30 minutes.

After carrying out the said re-reaction step, the said method of improving the sensitivity of competitive immunoassay further includes the following steps:
Chromogen reaction: adding chromogenic substrate solution into the said re-reaction product;
Reaction termination: sulfuric acid solution may be added to terminate the reaction;
Detection: the OD values of the said reaction product may be measured by a microplate reader.

The said method of improving the sensitivity of competitive immunoassay, is suitable for enzyme-linked immunosorbent assay (ELISA), time-resolved fluoroimmunoassay (Tr-FIA), radioimmunoassay (RIA), chemiluminescence immune assay (CLIA), magnetic chemiluminescence immune assay (MCLIA), protein microarray analysis, liquichip-assay, and other immunological analysis techniques. The present invention owns an easy operation method and a low cost, while does not require expensive equipments or professional technological personnel, furthermore, it owns a high sensitivity and adapts to a plurality of immunoassay methods.

### EMBODIMENT 1

### If the Present Pre-Reaction Mode Could Improve the Sensitivity. (Take the Detection of Ciprofloxacin with ELISA for Example)

Coat the ELISA plate with ciprofloxacin BSA conjugate, 1 μg/mL, 100 μL per well, blocking overnight at 4° C. Then block with 3% skimmed milk powder the following day. Prepare ciprofloxacin standards, 0 ppb, 1 ppb, 3 ppb, 9 ppb, 27 ppb, 81 ppb with the wash buffer (PBST).

The experiment is divided into two groups.

Group 1: pre-incubate 60 μL standards and 60 μL diluted ciprofloxacin antibody for 20 minutes at 37° C.; then take 100 μL reaction product and add to the ELISA plate covering with ciprofloxacin conjugate, react for 30 minutes at 37° C.; wash the plate, add HRP labeled secondary antibody, react for 30 minutes at 37° C., wash the plate; add chromogenic agent 100 μL, react for 15 minutes at 37° C.; and finally add 50 μL termination solution, then measure the OD450 with a microplate reader.

Group 2: add into the ELISA plate covering with ciprofloxacin conjugate with 50 μL standard and 50 μL antibody, in the sequence; react for 30 minutes at 37° C., wash the plate; add HRP labeled secondary antibody, react for 30 minutes at 37° C., wash the plate; add chromogenic agent 100 μL, react for 15 minutes at 37° C.; and finally add 50 μL termination solution, then measure OD450 with a microplate reader.

Measurement results are shown in the following table. It is possible to be seen that, comparing group 1 to group 2, the detection sensitivity did not get improved.

| | Ciprofloxacin concentration (ppb) | | | | | |
|---|---|---|---|---|---|---|
| | 0 | 1 | 3 | 9 | 27 | 81 |
| Group 1: OD450 | 2.072 | 1.975 | 1.632 | 1.448 | 0.924 | 0.483 |
| Group 2: OD450 | 2.036 | 1.904 | 1.643 | 1.203 | 0.952 | 0.523 |

### EMBODIMENT 2

### The Affections of Different Re-Reaction Time. (Take the Detection of Ciprofloxacin with ELISA for Example)

Coat the ELISA plate with ciprofloxacin BSA conjugate, 1 μg/mL, 100 μL per well, blocking overnight at 4° C. Then block with 3% skimmed milk powder the following day. Prepare ciprofloxacin standards, 0 ppb, 1 ppb, 5 ppb, and 10 ppb with wash buffer.

The experiment is divided into 5 groups, each group takes 100 μL standard and 100 μL ciprofloxacin antibody, pre-react for 20 minutes at 37° C. in the microplates, then add to ciprofloxacin ELISA plates:

Take 120 μL pre-reaction product to the ciprofloxacin ELISA plates from group 1, group 2, group 3 and group 4, and resume reacting for 30 minutes, 20 minutes, 10 minutes and 5 minutes respectively; wash the plate; add HRP labeled secondary antibody, react for 30 minutes at 37° C., wash the plate; add chromogenic agent 100 μL, react for 15 minutes at 37° C.; and finally add 50 μL termination solution, then measure the OD450.

Take 20 μL pre-reaction solution of antibody and standard from group 5 and add to the ciprofloxacin ELISA plate, react for 1 minute, discard the liquid in the well, and pat dry on absorbent paper, then add 20 μL mixture solution and react for 1 minute, repeat for 6 times altogether; wash the plate; add HRP labeled secondary antibody, react for 30 minutes at 37° C., wash the plate; add chromogenic agent 100 μL, react for 15 minutes at 37° C.; and finally add 50 μL termination solution, then measure the OD450.

Measurement results are shown in the following table. It is possible to be seen from the table that, competitive reaction with the solid phase antigen in the ELISA plate in a short time after the pre-reaction between the antibody and the standard, can significantly improve the sensitivity of the reaction, while the IC50 may be decreased to 1 ppb from 10 ppb.

| Ciprofloxacin | Time | | | | |
|---|---|---|---|---|---|
| concentration (ppb) | 30 min | 20 min | 10 min | 5 min | 1 min, 6 times |
| 0 | 2.479 | 2.195 | 2.188 | 1.74 | 1.778 |
| | 2.245 | 1.904 | 2.037 | 1.604 | 1.727 |
| 1 | 2.036 | 1.884 | 1.517 | 1.21 | 0.863 |
| | 1.989 | 1.874 | 1.488 | 1.189 | 0.813 |
| 5 | 1.501 | 1.342 | 1.003 | 0.706 | 0.412 |
| | 1.499 | 1.343 | 0.984 | 0.664 | 0.372 |
| 10 | 1.182 | 1.101 | 0.749 | 0.485 | 0.332 |
| | 1.269 | 1.204 | 0.894 | 0.565 | 0.302 |

### EMBODIMENT 3

### The Affections of Different Pre-Reaction Time. (Take the Detection of Ciprofloxacin with ELISA for Example)

Coat the ELISA plate with ciprofloxacin BSA conjugate, 1 μg/mL, 100 μL per well, blocking overnight at 4° C. Then block with 3% skimmed milk powder the following day. Prepare ciprofloxacin standards, 0 ppb, 1 ppb, 5 ppb, and 10 ppb with wash buffer.

The experiment is divided into 4 groups, each group takes 100 μL standard and 100 μL ciprofloxacin antibody, pre-incubate for 5 minutes, 10 minutes, 15 minutes and 20 minutes at 37° C. in the microplates respectively, then take 20 μL mixture solutions of antibody and standard and add to the ciprofloxacin ELISA plate, react for 1 minute, discard the liquid in the well, and pat dry on absorbent paper, then add 20 μL mixture solution and react for 1 minute, repeat for a total 6 times; wash the plate; add HRP labeled secondary antibody, react for 30 minutes at 37° C., wash the plate; add chromogenic agent 100 μL, react for 15 minutes at 37° C.; and finally add 50 μL termination solution, then measure the OD450.

Measurement results are shown in the following table. It is possible to be seen from the table that, over 5-minute pre-incubation time does not affect the results a lot, taking account of the time differences for sampling, the pre-incubation time could be extended to 20 minutes.

| | |
|---|---|
| Ciprofloxacin | Pre-incubation time |

| concentration (ppb) | 5 min | 10 min | 15 min | 20 min |
|---|---|---|---|---|
| 0 | 0.812 | 0.851 | 0.842 | 0.831 |
| | 0.789 | 0.863 | 0.823 | 0.811 |
| 1 | 0.337 | 0.385 | 0.399 | 0.434 |
| | 0.337 | 0.353 | 0.401 | 0.439 |
| 5 | 0.17 | 0.175 | 0.193 | 0.213 |
| | 0.169 | 0.187 | 0.206 | 0.22 |
| 10 | 0.133 | 0.136 | 0.149 | 0.178 |
| | 0.15 | 0.152 | 0.153 | 0.169 |

### EMBODIMENT 4

### Detecting Ractopamine with ELISA

Preparing the Ractopamine microplate: Ractopamine coating antigen is added to the microplate, 100 μL/well, react for 2 hours at 37° C.; discard the liquid in the well; add blocking solution into the microplate, 100 μL/well, blocking for 1 hour at 37° C.; wash 3 times with wash buffer, leave for reservation. Prepare Ractopamine standards with concentrations of 0 ppb, 0.05 ppb, 0.15 ppb, 0.45 ppb, 1.35 ppb, 4.05 ppb respectively, with wash buffer.

Divide into two groups:

Group 1: add 60 μL the standard and the Ractopamine antibody respectively into a new microplate, pre-react for 20 minutes; take 20 μL mixture solution of antibody and standard into the Ractopamine microplate, react for 1 minute, discard the liquid in the well, and pat dry on absorbent paper, then add 20 μL mixture solution and react for 1 minute, repeat for 5 times altogether, the pre-reaction product added into each well is 100 μL altogether; add HRP labeled secondary antibody, 100 μL per well, react for 45 minutes at 37° C.; wash the plate; add chromogenic agent 100 μL, react for 15 minutes at 37° C.; and finally add 50 μL termination solution, then measure the OD450.

Group 2: add 60 μL the standard and the Ractopamine antibody respectively into a new microplate, pre-react for 20 minutes; take 100 μL mixture solution of antibody and standard into the Ractopamine microplate, react for 30 minutes, discard the liquid in the well, and pat dry on absorbent paper; wash the plate; add HRP labeled secondary antibody, 100 μL per well, react for 45 minutes at 37° C., wash the plate; add chromogenic agent 100 μL, react for 15 minutes at 37° C.; and finally add 50 μL termination solution, then measure the OD450.

Measurement results are shown in the following table. It is possible to be seen that, group 1 can significantly improve the detection sensitivity.

| | Ciprofloxacin concentration (ppb) | | | | | |
|---|---|---|---|---|---|---|
| | 0 | 0.05 | 0.15 | 0.45 | 1.35 | 4.05 |
| Group 1: OD | 1.472 | 1.175 | 0.803 | 0.517 | 0.324 | 0.183 |
| Group 2: OD | 1.636 | 1.584 | 1.391 | 1.003 | 0.602 | 0.323 |

### EMBODIMENT 5

### Application in the Chemiluminescence Immunoassay (Take the Detection of Chloramphenicol as an Example)

1) Preparation of the chloramphenicol microplate: add chloramphenicol-BSA conjugate into the ELISA plate, 100 μL per well, overnight at 4° C., wash 3 times with a separation time of 3 minutes each, pat dry; add 5% skimmed milk powder 130 μL per well, block for 1 hour at 37° C., pat dry; wash 3 times with wash buffer, pat dry, leave for reservation. Prepare the chloramphenicol standard solutions with wash buffer: 0 ppb, 0.01 ppb, 0.03 ppb, 0.09 ppb, 0.27 ppb, 0.81 ppb.
2) Mix the standard and the chloramphenicol Mcab in equal volumes, incubate for 20 minutes at 37° C.; then transfer the reaction product into chloramphenicol microplate;
Group 1: transfer the reaction product into ELISA plate, 20 μL/well, reaction for 1 minute at 37° C., discard liquid in the well, repeat the operation 5 times, and the pre-reaction product added to each well is total 100 μL;
Group 2: one-time transfer the whole 100 μL reaction product into the chloramphenicol microplate, reacting for 20 minute at 37° C.;
3) Wash 3 times; add HRP labeled secondary antibody, 100 μL per well, react for 45 minutes at 37° C.; wash 5 times; after adding luminous substrate, chemiluminescence measuring instrument is applied to measure the luminous intensity.

The following table shows the data collected in the test.

| | Concentration of chloramphenicol standard(ppb) | | | | | |
|---|---|---|---|---|---|---|
| | 0 | 0.01 | 0.03 | 0.09 | 0.27 | 0.81 |
| Group 1 | 0.99 | 0.88 | 0.66 | 0.49 | 0.21 | 0.11 |
| (B/B0) | 0.97 | 0.89 | 0.64 | 0.48 | 0.23 | 0.17 |
| Group 2 | 0.99 | 0.96 | 0.88 | 0.69 | 0.46 | 0.28 |
| (B/B0) | 0.98 | 0.94 | 0.89 | 0.69 | 0.48 | 0.27 |

From the data listed above, it has shown that the sensitivity of detecting chloramphenicol adopting the method described in the present invention is higher than that of regular detection methods.

It should be understood that, the application of the present invention is not limited to the above examples listed. Ordinary technical personnel in this field can improve or change the applications according to the above descriptions, all of these improvements and transforms should belong to the scope of protection in the appended claims of the present invention.

What is claimed is:

1. A method of improving the sensitivity of competitive immunoassay, wherein, the steps of the said method of improving the sensitivity of competitive immunoassay comprise:
pre-reaction: a substances for detection pre-react with a receptors/antibodies;
re-reaction: a reaction products collected from the pre-reaction process react with a second molecules locating on a solid phase and attending the competitive reactions;
during the said re-reaction process, the reaction product collected from the pre-reaction process is divided into one or more portions, then reacting with the said second molecules respectively.

2. The said method of improving the sensitivity of competitive immunoassay, according to claim 1, wherein, during the said re-reaction process, the said pre-reaction products react with the said second molecules in a way of flowing through the said solid phase surfaces.

3. The said method of improving the sensitivity of competitive immunoassay, according to claim 2, wherein, during the said re-reaction process, the reaction time between each portion of the pre-reaction product and the said second molecules is within 20 minutes.

4. The said method of improving the sensitivity of competitive immunoassay, according to claim 2, wherein, during the said re-reaction process, the reaction time between each portion of the pre-reaction product and the said second molecules is within 5 minutes.

5. The said method of improving the sensitivity of competitive immunoassay, according to claim 1, wherein, after the reaction between each portion of the pre-reaction product and the said second molecules is finished, a post reaction liquid is removed.

6. The said method of improving the sensitivity of competitive immunoassay, according to claim 1, wherein, the reaction temperature of the said pre-reaction process is between 0° C. and 65° C., and the reaction time lasts for 0-3 hours.

7. The said method of improving the sensitivity of competitive immunoassay, according to claim 6, wherein, the reaction time of the said pre-reaction process lasts for 30 minutes.

8. The said method of improving the sensitivity of competitive immunoassay, according to claim 1, wherein, the said second molecules refer to the conjugates formed by small molecules participating in the competitive reactions and the carrier proteins.
